(19) United States
(12) Patent Application Publication
(10) Pub. No.: US 2007/0142699 A1
(43) Pub. Date: Jun. 21, 2007

(54) Methods and implantable apparatuses for treating an esophageal disorder such as gastroesophageal reflux disease

(75) Inventor: Sally D. Jandrall, Seattle, WA (US)

Correspondence Address: PERKINS COIE LLP;PATENT-SEA, P.O. BOX 1247, SEATTLE, WA, 98111-1247, US

(73) Assignee: AcousTx Corporation

(21) Appl. No.: 11/305,459

(22) Filed: Dec. 16, 2005

(51) Int. Cl. A61F 2/00

(52) U.S. Cl. 600/37

(57) ABSTRACT

Methods and implantable apparatuses for treating esophageal disorders such as gastroesophageal reflux disease in patients are disclosed herein. In one embodiment, a method includes inserting a constricting member into a patient, and positioning the constricting member around at least a portion of the stomach of the patient with a section of the constricting member positioned to exert a force on the stomach and/or gastroesophageal junction that is expected to recalibrate the cardia and improve the competence of the lower esophageal sphincter.

### TECHNICAL FIELD

The present invention is related to methods and implantable apparatuses for treating an esophageal disorder such as gastroesophageal reflux disease.

### BACKGROUND

Gastroesophageal reflux disease (GERD) is a common gastroesophageal disorder in which the stomach contents reflux into the lower esophagus due, in part, to a dysfunction of the lower esophageal sphincter (LES). The antireflux barrier in normal individuals is a highly competent structure that withstands enormous pressures without allowing reflux. For example, a 250-lb wrestler can land on his opponent's abdomen without causing the opponent to vomit. The LES maintains a resting pressure higher than the pressure in the adjacent esophagus or stomach. This high pressure zone separates the gastric cavity from the esophageal lumen. Stomach contents are usually acidic. Hence, gastric reflux into the lower esophagus due to LES dysfunction is potentially injurious to the esophagus resulting in a number of possible complications of varying medical severity. The reported incident of GERD in the U.S. is as high as 10% of the population.

Acute symptoms of GERD include heartburn, laryngeal problems, pulmonary disorders and chest pain. On a chronic basis, GERD subjects the esophagus to ulceration and inflammation, and may result in more severe complications including esophageal obstruction, acute and/or chronic blood loss, and cancer. In fact, the increasing incidence of adenocarcinoma of the esophagus, which is rising faster than any other cancer, is believed to be directly linked to the increasing incidence and severity of GERD. GERD typically requires lifelong medical therapy or surgery for the management of patients with frequent symptoms.

Current drug therapy for GERD includes proton-pump inhibitors (PPI) that reduce stomach acid secretion and other drugs which may completely block stomach acid production. However, while pharmacologic agents often provide symptomatic relief and allow esophagitis to heal, they do not address the underlying cause of LES dysfunction. Drug therapy is also expensive, and may impair digestion.

A number of invasive procedures have been developed in an effort to correct the dysfunctional LES in patients with GERD. The role of surgery is to restore the function of the incompetent antireflux barrier. One such procedure, gastric fundoplication, involves wrapping the gastric fundus, partially or completely around the lower esophagus. This anatomic rearrangement results in the creation of an increased zone of high intragastric pressure following meals that can prevent reflux of gastric contents into the esophagus. However, the gastroesophageal junction is more than a flaccid rubber tube; in order for a gastric fundoplication to be effective, it must restore several aspects of the dysfunctional anatomy and physiology that exists in patients with GERD. First, in those with a hiatal hernia in which the LES has moved above the diaphragmatic hiatus into the chest where pressure is less than the abdomen, the operation must restore the position of the GE junction and LES below the diaphragm. Second, the esophageal crura must be approximated and the GE junction secured below the diaphragm to prevent recurrent herniation and migration of the LES above the diaphragm again. Thirdly, the fundoplication must also produce a recalibration of the cardia. Calibration of the cardia narrows the angle of His and improves the coincidence of the mucosal seal and the size of the mucosal contact zone. Classic antireflux surgery does not, however, always restore all of these aspects of the dysfunctional anatomy, which could explain why antireflux surgery fails in a significant number of patients, especially those with long-segment and complicated Barrett's esophagus. Although gastric fundoplication has a high rate of success, it is an open abdominal procedure with the usual risks of abdominal surgery including: postoperative infection, herniation at the operative site, internal hemorrhage, and perforation of the esophagus or the cardia.

Recently, gastric fundoplication has been able to be performed using minimally invasive surgical techniques. This procedure involves essentially the same steps as an open gastric fundoplication with the exception that surgical manipulation is performed through several small incisions by way of surgical trocars inserted at various positions in the abdomen. This less invasive surgical approach is capable of restoring the LES similar to the open operation but patients recover from surgery quicker and with less discomfort.

As an alternative to open or minimally invasive surgery, a number of endoluminal techniques have been recently developed as treatment options for GERD. These techniques are even less invasive than the laparoscopic gastric fundoplication in that devices are inserted through the mouth into the esophagus to reach the area of the LES. One such technique, disclosed in U.S. Pat. No. 5,088,979, uses an invagination device containing a number of wires and needles which are in a retracted position inserted transorally into the esophagus. Once positioned at the LES, the needles are extended to engage the esophagus and fold the attached esophagus beyond the gastroesophageal junction. A remotely operated stapling device, introduced percutaneously through an operating channel in the stomach wall, is actuated to fasten the invaginated gastroesophageal junction to the surrounding involuted stomach wall.

Another device is disclosed in U.S. Pat. No. 5,676,674. In this procedure, invagination is performed with a jaw-like device, and the invaginated gastroesophageal junction is fastened to the fundus of the stomach with a transoral approach using a remotely operated fastening device, eliminating the need for an abdominal incision. However, this procedure is still traumatic to the LES and presents the post-operative risks of gastroesophageal leaks, infection, and foreign body reaction, the latter sequela resulting when foreign materials such as surgical staples are implanted in the body.

Curon Medical has developed a radio-frequency ablation device (disclosed in U.S. Pat. No. 6,846,312) that is also delivered to the gastroesophageal junction transorally. The device first penetrates the esophagus with RF electrodes arranged in a circular fashion. RF energy is delivered into the muscular tissues to cause a tightening of the LES through the generation of lesions in the tissue. There have been a number of major complications resulting from this device, and its effectiveness is debated.

There are also several device approaches based on the idea of injecting bulking agents into the LES. They suffer from short-term effectiveness. Enteryx (now owned by Boston Scientific Corp.) is the only FDA approved device based on this approach. Each injection of the implanted material is performed with the aid of fluoroscopy to ensure accurate deep mural placement of the implant. Concomitant endoscopic imaging is utilized to avoid misdirected large volume submucosal implants, which will ulcerate the esophageal mucosa and slough off if not placed deep within the muscle.

### BRIEF DESCRIPTION OF THE DRAWINGS

FIG. 1 is a schematic representation of a portion of the human anatomy including an esophagus, a stomach, and a gastroesophageal junction (or cardia).

FIG. 2 is a schematic cross-sectional view of a gastroesophageal junction taken generally along the line A-A of FIG. 1 in an individual with a normal cardia.

FIGS. 3A-3D are schematic representations of the expected orientation and operation of sling and clasp fibers in an individual with a normal cardia.

FIG. 4 is a schematic cross-sectional view of a gastroesophageal junction taken generally along the line A-A of FIG. 1 in an individual with a dilated cardia.

FIGS. 5A-5D are schematic representations of the expected orientation and operation of sling and clasp fibers in an individual with a dilated cardia.

FIG. 6 is a schematic representation of a constricting member implanted relative to the stomach of a patient with GERD in accordance with one embodiment of the invention.

FIG. 7 is a schematic representation of a plurality of constricting members implanted relative to the stomach of a patient with GERD in accordance with another embodiment of the invention.

FIG. 8 is a schematic representation of a constricting member implanted relative to the stomach of a patient with GERD in accordance with another embodiment of the invention.

FIG. 9 is a schematic representation of a plurality of constricting members implanted relative to the stomach of a patient with GERD in accordance with another embodiment of the invention.

FIG. 10 is a schematic representation of a plurality of constricting members implanted relative to the stomach of a patient with GERD in accordance with another embodiment of the invention.

FIG. 11 is a schematic representation of a member implanted relative to the stomach of a patient with GERD in accordance with another embodiment of the invention.

FIG. 12 is a schematic representation of a constricting member implanted relative to the stomach of a patient with GERD in accordance with another embodiment of the invention.

### DETAILED DESCRIPTION

A. Overview

The present invention is directed toward implantable apparatuses and methods of treating esophageal disorders such as gastroesophageal reflux disease. One embodiment of a method includes inserting a constricting member into a patient and positioning the constricting member around at least a portion of the stomach such that a section of the constricting member exerts a force on the stomach and/or gastroesophageal junction that is expected to recalibrate and restore the cardia and improve the competence of the lower esophageal sphincter. The constricting member includes a first portion and a second portion. In several applications, the first portion is positioned at the angle of His and the second portion is positioned at the greater curvature of the stomach. In other applications, the first portion can be positioned at the junction between the esophagus and the lesser curvature of the stomach and the second portion can be positioned at the greater curvature of the stomach.

In one aspect of this embodiment, the method further includes inserting a second constricting member into the patient, and positioning the second constricting member around the stomach. The second constricting member includes a first portion and a second portion. The first portion of the second constricting member can be positioned at the angle of His or at the junction between the esophagus and the lesser curvature of the stomach, and the second portion of the second constricting member can be positioned at the greater curvature of the stomach. As such, the first and second constricting members are each generally aligned with at least some of the sling or clasp fibers in the lower esophageal sphincter to augment the operation of these fibers.

In another embodiment, a method includes implanting a band in a patient with the band positioned about a section of the stomach. A portion of the band is positioned at the angle of His and exerts a force that augments the sling fibers. In other embodiments, the portion of the band can be positioned at the junction between the esophagus and the lesser curvature of the stomach. In either case, the band is expected to recalibrate and restore the cardia and improve the competence of the lower esophageal sphincter.

Another aspect of the invention is directed to apparatuses for treating esophageal disorders. In one embodiment, an apparatus comprises a band including a first portion configured to conform to the angle of His in the patient and a second portion configured to be positioned along a section of the stomach and overlay at least some of the sling fibers. As such, the band is expected to recalibrate and restore the cardia and improve the competence of the lower esophageal sphincter.

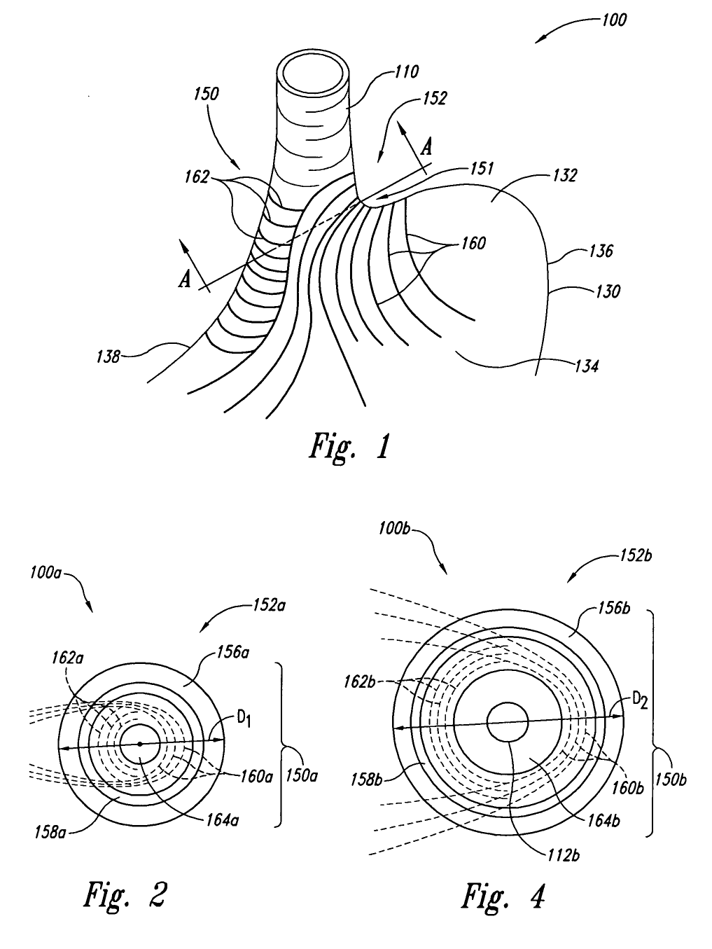

The following disclosure describes apparatuses and methods of treating esophageal disorders such as gastroesophageal reflux disease in patients. Unless the term “or” is expressly limited to mean only a single item exclusive from the other items in reference to a list of two or more items, then the use of “or” in such a list is to be interpreted as including (a) any single item in the list, (b) all of the items in the list, or (c) any combination of items in the list. Certain details are set forth in the following description and in FIGS. 1-12 to provide a thorough understanding of various embodiments of the invention. Other details describing the operation, anatomy, and physiology of portions of the gastrointestinal tract are not set forth in the following disclosure to avoid unnecessarily obscuring the description of various embodiments of the invention.

Many of the details, positions, and other features shown in the figures are merely illustrative of particular embodiments of the invention. Accordingly, other embodiments can have other details, positions, and/or features without departing from the spirit or scope of the present invention. In addition, further embodiments of the invention may be practiced without several of the details described below, or various aspects of any of the embodiments described below can be combined in different combinations.

B. Gastrointestinal Tract and Gastroesophageal Reflux Disease

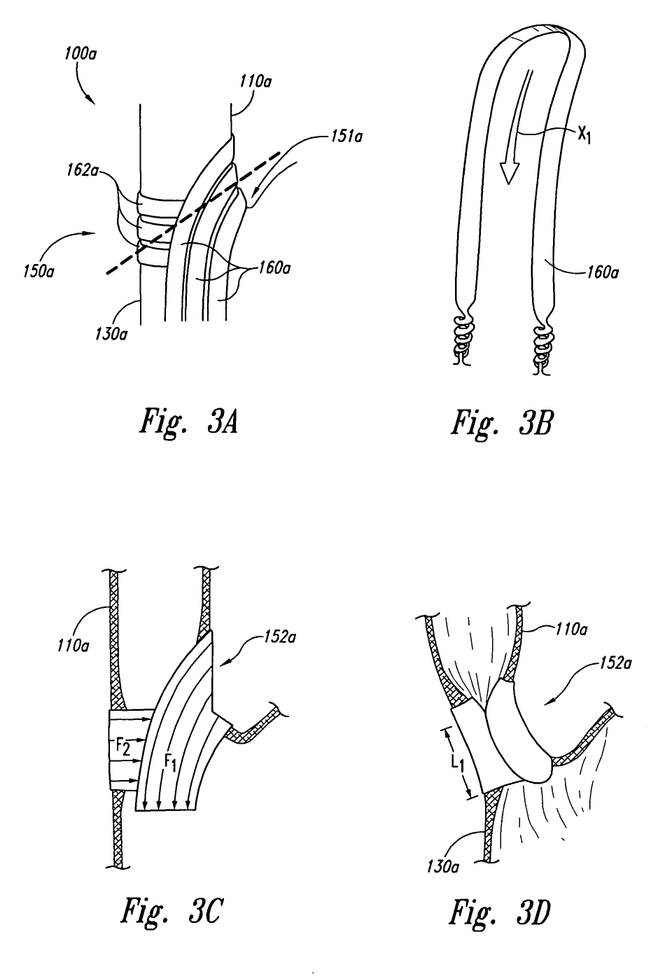

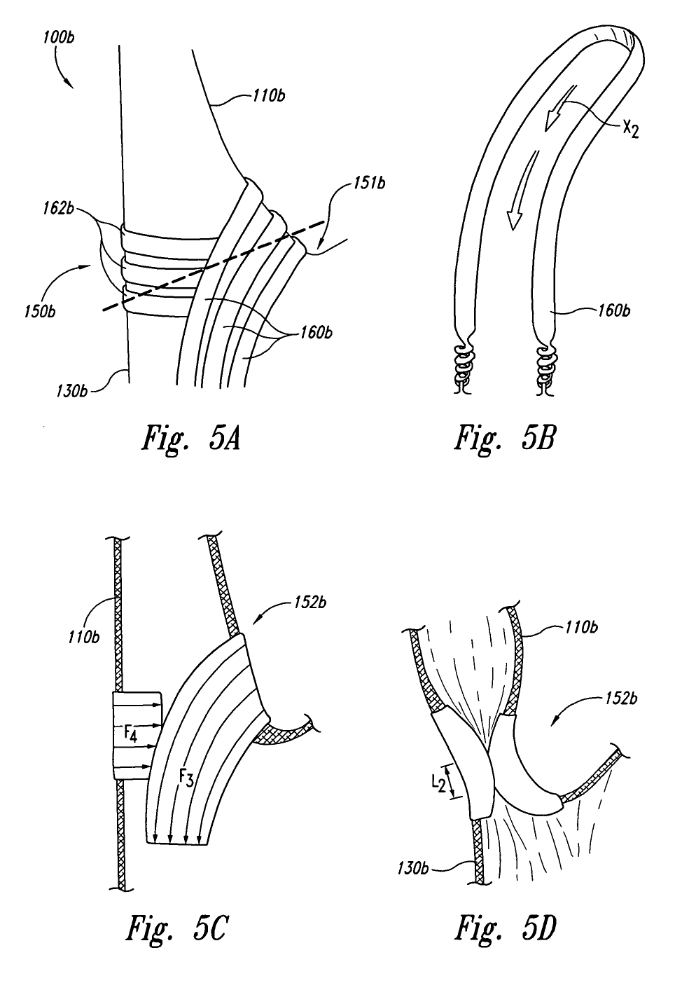

FIG. 1 is a schematic representation of an internal portion of an individual 100 including an esophagus 110, a stomach 130, and a gastroesophageal junction (or cardia) 150 between the esophagus 110 and the stomach 130. The terms gastroesophageal junction and cardia are used interchangeably herein. The stomach 130 has a fundus 132 adjacent to the cardia 150, a body 134 adjacent to the fundus 132, a greater curvature 136 extending around the body 134 and a portion of the fundus 132, and a lesser curvature 138 extending around the body 134 and ending at the gastroesophageal junction 150. The gastroesophageal junction 150 has an angle of “His” 151 between the esophagus 110 and the stomach 130 and a lower esophageal sphincter 152 at the end of the esophagus 110. The lower esophageal sphincter 152 is comprised of two muscular groups, namely gastric sling fibers 160 (shown in the figures as lines) and semicircular clasp fibers 162 (shown in the figures as lines). The sling fibers 160 have a generally oblique orientation and extend from the body 134 of the stomach 130 over the angle of His 151 to form a sling-like structure. The clasp fibers 162 are generally semicircular fibers positioned generally transverse to the sling fibers 160. The sling and clasp fibers 160 and 162 operate together to form the lower esophageal sphincter 152 and maintain the high pressure zone that confines the gastric environment to the stomach. The operation of the sling and clasp fibers 160 and 162 is described in greater detail below with reference to FIGS. 3A-3D and 5A-5D.

The lower esophageal sphincter (LES) 152 selectively inhibits gastric acid and other stomach contents from passing into the lower esophagus 110. In some people, however, the LES 152 becomes mechanically incompetent or dysfunctional, resulting in Gastroesophageal Reflux Disease (GERD). A dysfunctional LES 152 occurs when there is a decrease in LES pressure, the coincidence of the mucosal seal is degraded, and the length of the high pressure zone shortens. There is a correlation between individuals with a dilated cardia 150 (or enlarged perimeter of the gastroesophageal junction) and the severity of GERD. Anatomic dilation of the cardia 152 implies a permanent morphologic change in the gastroesophageal junction, provoked of necessity by an alteration in the architecture or arrangement of the muscular components that shape it. For example, chronic dilation of the cardia 150 alters the function of the sling and clasp fibers 160 and 162. Specifically, dilation of the cardia 150 implies elongation of the sling and clasp muscular fibers 160 and 162, and alteration in their relative angulation and arrangement. The length-tension properties of the elongated muscle fibers are degraded, resulting in reduced LES pressure. Moreover, because of the altered orientation of the sling and clasp fibers 160 and 162, the fibers 160 and 162 may not effectively interact, which also reduces the LES pressure. In addition, alteration of the relative orientation of the sling and clasp fibers 160 and 162 reduces the contact area (the mucosal seal) and shortens the high pressure zone such that the LES 152 is easier to open. Furthermore, the enlarged perimeter of the gastroesophageal junction 150 effectively reduces the LES pressure because less force is required to open the larger diameter (Law of La Place). Moreover, the angle of His 151 may also be increased. Thus, the closing pressure is impaired, and a mechanically defective LES 152 results.

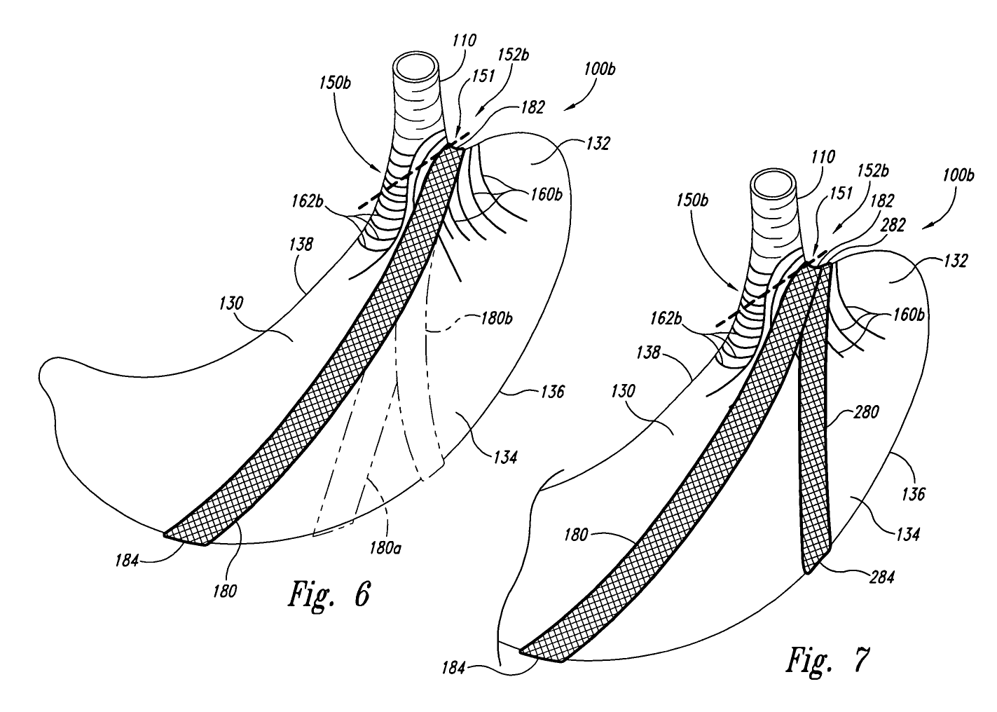

Although a dilated cardia 150 is not the origin of GERD, it represents a point at which the LES 152 becomes mechanically incompetent. Augmenting and/or imitating the normal tension applied by competent sling and/or clasp fibers 160a and/or 162a to correct for the misalignment and altered state of the fibers 160b and/or 162b reduces the perimeter of the cardia 150. By the Law of La Place, a reduced perimeter effectively increases the LES pressure. Reduction in the perimeter of the cardia 150 should also recalibrate the cardia 150 by narrowing the angle of His 151. Moreover, augmenting and/or imitating the normal tension applied by competent sling and/or clasp fibers 160a and/or 162a to correct for the misalignment and altered state of the fibers 160b and/or 162b is expected to increase (a) the LES pressure, (b) the coincidence of the mucosal contact area, and (c) the length of the high pressure zone. Therefore, the above-described alterations improve the mechanical function or competence of the LES 152. Several apparatuses for augmenting and/or imitating the normal tension applied by competent sling and/or clasp fibers 160a and/or 162a to correct for the misalignment and altered state of the fibers 160b and/or 162b are discussed in detail below with regard to FIGS. 6-12.

FIG. 2 is a schematic cross-sectional view of a gastroesophageal junction 150a taken generally along the line A-A of FIG. 1 in an individual 100a with a normal cardia 150a. The gastroesophageal junction 150a includes a plurality of outer layers 156a, a longitudinal muscle layer 158a radially inward of the outer layers 156a, a plurality of sling and clasp fibers 160a and 162a radially inward of the longitudinal muscle layer 158a, and a mucosa/submucosa layer 164a radially inward of the sling and clasp fibers 160a and 162a. In the normal cardia 150a, the sling and clasp fibers 160a and 162a have normal length-tension properties and are properly positioned relative to each other for effectively operating together and forming a competent LES 152a with a zone of high pressure of normal length and pressure. The normal cardia 150a has a diameter D₁ of approximately 2 centimeters in a healthy adult.

FIGS. 3A-3D are schematic representations of the expected orientation and operation of the sling and clasp fibers 160a and 162a in the individual 100a with the normal cardia 150a. For example, FIG. 3A illustrates the normal orientation of sling and clasp fibers 160a and 162a at the gastroesophageal junction 150a. Specifically, the sling fibers 160a have an oblique orientation and extend from one side of the stomach 130a, over the angle of His 151a, to the other side of the stomach 130a. The clasp fibers 162a have a lateral orientation and a semicircular configuration such that they do not extend completely around the gastroesophageal junction 150a. The sling fibers 160a are positioned generally on one side of the gastroesophageal junction 150a, and the clasp fibers 162a are positioned generally on the other side of the gastroesophageal junction 150a such that the fibers 160a and 162a cooperate to form a competent LES 152a.

FIG. 3B illustrates a force vector X₁ representing the force exerted by the individual sling fibers 160a. The force vector X₁ of the individual sling fibers 160a has a generally vertical orientation. FIG. 3C illustrates a combined force F₁ exerted by the individual sling fibers 160a across a first displacement area and a combined force F₂ exerted by the individual clasp fibers 162a across a second displacement area. The mucosal seal (or closure area) is reached at the intersection of the first and second displacement areas. FIG. 3D illustrates the competent lower esophageal sphincter 152a in the contracted position. Because the sling and clasp fibers 160a and 162a have normal force vectors, the closure area or high pressure zone formed by the sling and clasp fibers 160a and 162a has a normal length L₁ and pressure. Specifically, the resting pressure in the competent lower esophageal sphincter 152a is typically 15-25 mmHg above the intragastric pressure as measured by conventional manometry techniques. This pressure, however, can vary throughout the day. The sling and clasp fibers 160a and 162a form a competent LES 152a and accordingly maintain a normal gastroesophageal pressure gradient.

FIG. 4 is a schematic cross-sectional view of a gastroesophageal junction 150b taken generally along the line A-A of FIG. 1 in an individual 100b with a dilated cardia 150b. When the cardia 150b is chronically dilated, the oblique sling fibers 160b are separated, elongated, and angulated, modifying their length-tension properties relative to normal sling fibers 160a. These changes result in reduced LES pressure, a smaller mucosal contact area, and a shorter high pressure zone. Consequently, the LES 152b is mechanically defective.

FIGS. 5A-5D are schematic representations of the expected orientation and operation of the sling and clasp fibers 160b and 162b in the individual 100b with a dilated cardia 150b. For example, FIG. 5A illustrates the altered orientation of the sling and clasp fibers 160b and 162b at the gastroesophageal junction 150b. Specifically, the sling and clasp fibers 160b and 162b are lengthened and misaligned such that the angle of His 151b may become obtuse. FIG. 5B illustrates a force vector X₂ representing the force exerted by the individual sling fibers 160b. The force vector X₂ of the lengthened and misaligned sling fiber 160b has a horizontal component and is oriented transverse to the force vector X₁ (FIG. 3B) of the normal sling fiber 160a. FIG. 5C illustrates a combined force F₃ exerted by the individual sling fibers 160b across a third displacement area and a combined force F₄ exerted by the individual clasp fibers 162b across a fourth displacement area. The mucosal seal is reached at the intersection of the third and fourth displacement areas. The mucosal seal, however, is smaller and the high pressure zone is shortened. Thus, the LES 152b is mechanically incompetent.

FIG. 5D illustrates the incompetent lower esophageal sphincter 152b in the contracted position. The mucosal seal formed by the sling and clasp fibers 160b and 162b has a relatively short length L₂ and/or low pressure due to the altered orientation and elongation of the sling fibers 160b and/or clasp fibers 162b. Consequently, the length-tension properties of the sling and clasp fibers 160b and 162b have been altered, and the LES pressure is reduced. Because of the reduced pressure and/or short length L₂ of the mucosal seal the lower esophageal sphincter 152b is mechanically incompetent.

C. Embodiments of Constricting Members for Treating Gastroesophageal Reflux Disease

FIGS. 6-12 illustrate a plurality of implantable devices wrapped around or otherwise implanted relative to the stomachs of patients in accordance with several embodiments of the invention. The implantable devices augment the function of the sling and/or clasp fibers 160b and/or 162b by exerting forces on the stomach 130 and/or gastroesophageal junction 150b that augment and/or imitate the normal tension applied by competent sling and/or clasp fibers 160a and/or 162a to correct for the misalignment and altered state of the fibers 160b and/or 162b. As a result, the implantable devices are expected to reduce the perimeter of the cardia 150, thereby improving the relationship of the sling and/or clasp fibers 160b and/or 162b and allowing a more normal interplay between the fibers 160b and/or 162b. Therefore, augmenting and/or imitating the normal tension applied by competent sling and/or clasp fibers 160a and/or 162a with the implantable devices increases the lower esophageal sphincter pressure, enlarges the mucosal seal, and lengthens the high pressure zone. As with a Nissen fundoplication, the mechanical function of the incompetent LES 152b is improved and a normal gastroesophageal pressure gradient is expected to be restored.

FIG. 6 is a schematic representation of a constricting member 180 implanted relative to the stomach 130 of a patient 100b with GERD in accordance with one embodiment of the invention. The illustrated constricting member 180 is implanted relative to the stomach 130 so that the member 180 is at least generally aligned with the sling fibers 160b. Specifically, the constricting member 180 includes a first portion 182 positioned at the angle of His 151 and a second portion 184 positioned at the greater curvature 136. As such, the constricting member 180 is positioned to exert a force on the stomach 130 and/or gastroesophageal junction 150b that augments and/or imitates the normal tension of competent sling fibers 160a to correct for the misalignment and altered state of the fibers 160b. In other embodiments, the first and second portions 182 and 184 can be positioned at different locations. For example, the second portion 184 can be positioned at other sections of the greater curvature 136 (such as in the constricting members 180a and 180b illustrated in broken lines).

The illustrated constricting member 180 is an elastic band with two ends that can be fastened together to secure the member 180 around the stomach 130. The elastic band is sized to exert the force required for at least partially restoring the gastroesophageal pressure gradient and enhancing the mucosal seal of the lower esophageal sphincter 152b. The constricting member 180, however, is not limited to being an elastic band. For example, the constricting member 180 can be an inflatable bladder connected to a reservoir and/or pump to selectively inflate the bladder and adjust the force exerted by the constricting member. In additional embodiments, the constricting member 180 can be an inelastic, rigid, or other suitable member for least partially restoring the gastroesophageal pressure gradient and enhancing the mucosal seal of the lower esophageal sphincter 152b. Moreover, although the illustrated constricting member 180 extends completely around the stomach 130, in other embodiments, the constricting member may extend around only a portion of the stomach 130. For example, the constricting member may include a first end sutured to a portion of the body 134 on one side of the stomach 130, a second end sutured to the body 134 on the other side of the stomach 130, and a portion between the first and second ends positioned at the angle of His 151.

One feature of the constricting member 180 illustrated in FIG. 6 is that the constricting member 180 applies a force on the stomach 130 and/or gastroesophageal junction 150b that augments and/or imitates the normal tension of competent sling fibers 160a to correct for the misalignment and altered state of the fibers 160b. Consequently, the constricting member 180 is expected to increase the lower esophageal sphincter pressure, enlarge the mucosal seal, and lengthen the high pressure zone. The improved mechanical function of the lower esophageal sphincter 152b is expected to reduce and/or eliminate the reflux of gastric acid into the esophagus and the associated symptoms of GERD. As such, the illustrated constricting member 180 provides a long-term solution to GERD that does not involve many of the risks of conventional treatments.

FIG. 7 is a schematic representation of an embodiment having first and second constricting members 180 and 280 implanted relative to the stomach 130 of a patient 100b with GERD in accordance with another embodiment of the invention. The position and configuration of the first constricting member 180 is described above with reference to FIG. 6. The second constricting member 280 is also positioned around the stomach 130 and generally aligned with the sling fibers 160b. Specifically, the second constricting member 280 includes a first portion 282 positioned at the angle of His 151 and a second portion 284 positioned at the greater curvature 136. In the illustrated embodiment, the first portions 182 and 282 of the first and second constricting members 180 and 280 partially overlap at the angle of His 151, and the second portions 184 and 284 of the first and second constricting members 180 and 280 are spaced apart along the greater curvature 136. In other embodiments, however, the first portions 182 and 282 can be spaced apart, and/or the second portions 184 and 284 may be at least partially overlapped. In either case, the first and second constricting members 180 and 280 exert a force on the stomach 130 and/or gastroesophageal junction 150b that augments and/or imitates the tension of competent sling fibers 160a to correct for the misalignment and altered state of the fibers 160b. An advantage of placing multiple constricting members around the stomach 130 is that the members can apply a force over a greater area of the stomach 130 and/or gastroesophageal junction 150b to at least partially restore the gastroesophageal pressure gradient and enhance the mucosal seal of the lower esophageal sphincter 152b.

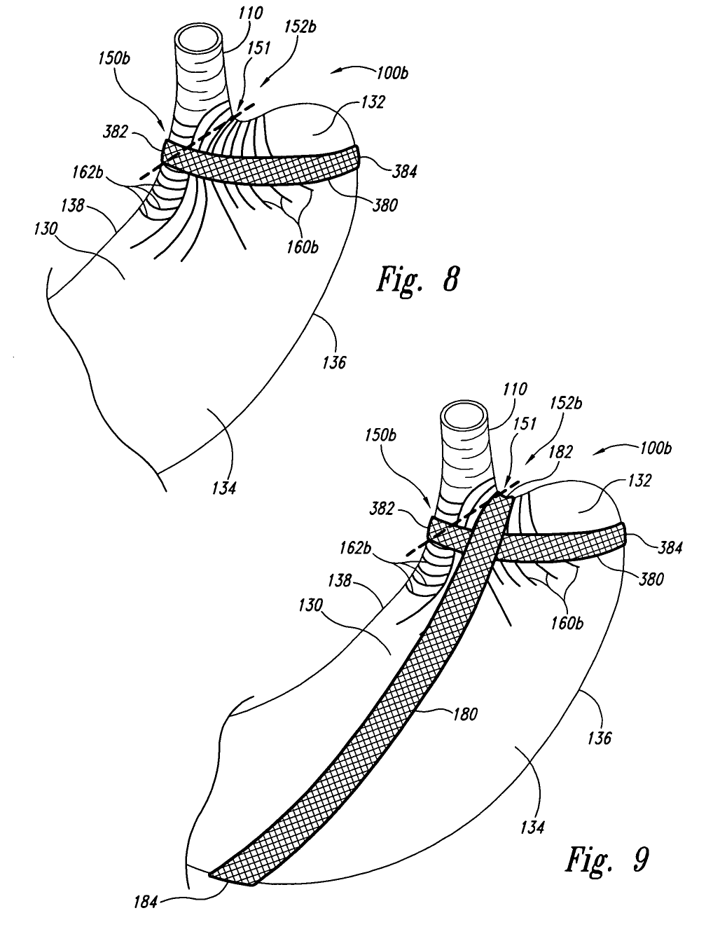

FIG. 8 is a schematic representation of a constricting member 380 implanted relative to the stomach 130 of a patient 100b with GERD in accordance with another embodiment of the invention. The constricting member 380 is placed around the stomach 130 so that the member 380 is generally aligned with the clasp fibers 162b. Specifically, the constricting member 380 includes a first portion 382 positioned at the gastroesophageal junction 150b and a second portion 384 positioned at the greater curvature 136 of the stomach 130. The second portion 384 can be positioned at the section of the greater curvature 136 that is proximate to the fundus 132, the body 134, and/or the junction between the fundus 132 and the body 134. In either case, the constricting member 380 applies a force on the stomach 130 and/or gastroesophageal junction 150b to augment and/or imitate the normal tension applied by competent clasp fibers 162a to correct for the misalignment and altered state of the fibers 162b. An advantage of this feature is that pressure from the constricting member 380 at least partially restores the gastroesophageal pressure gradient and enhances the mucosal seal of the lower esophageal sphincter 152b.

FIG. 9 is a schematic representation of first and second constricting members 180 and 380 implanted relative to the stomach 130 of a patient 100b with GERD in accordance with another embodiment of the invention. The position and configuration of the first constricting member 180 are described above with reference to FIG. 6. The position and configuration of the second constricting member 380 are described above with reference to FIG. 8. As such, the first constricting member 180 is generally aligned with the sling fibers 160b, and the second constricting member 380 is generally aligned with the clasp fibers 162b. The first and second constricting members 180 and 380 work together to exert a force on the stomach 130 and/or gastroesophageal junction 150b that augments and/or imitates the tension of competent sling and clasp fibers 160a and 162a to correct for the misalignment and altered state of the fibers 160b and 162b.

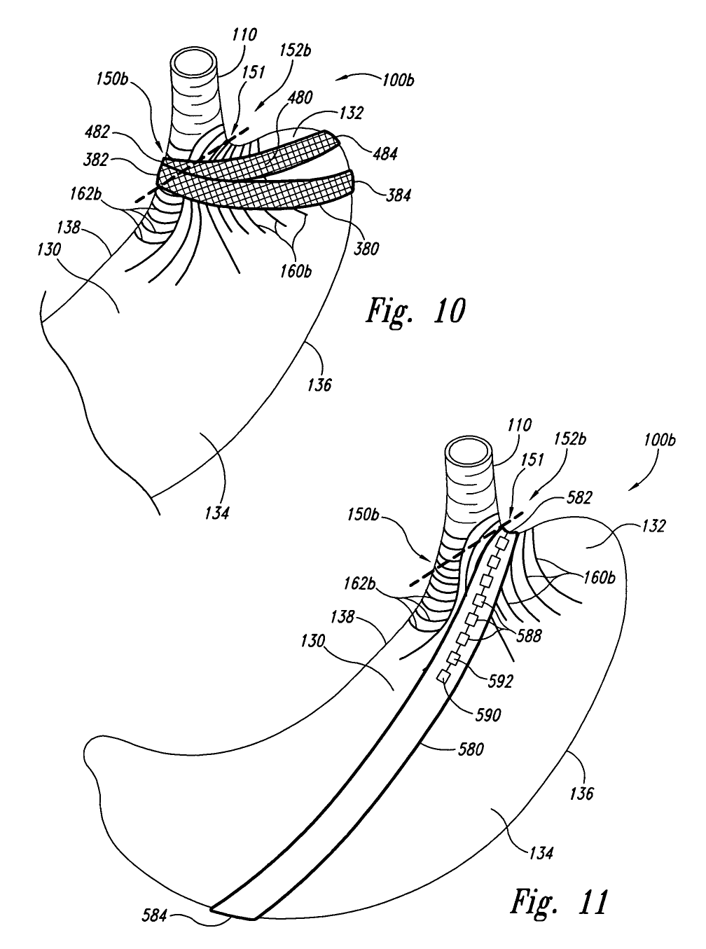

FIG. 10 is a schematic representation of first and second constricting members 380 and 480 implanted relative to the stomach 130 of a patient 100b with GERD in accordance with another embodiment of the invention. The position and configuration of the first constricting member 380 are described above with reference to FIG. 8. The second constricting member 480 is also positioned around the stomach 130 and generally aligned with the clasp fibers 162b. Specifically, the second constricting member 480 includes a first portion 482 positioned at the gastroesophageal junction 150b and a second portion 484 positioned at the greater curvature 136 of the stomach 130. The second portion 484 can be positioned at the section of the greater curvature 136 that is proximate to the fundus 132, the body 134, and/or the junction between the fundus 132 and the body 134. Although in the illustrated embodiment, the first portions 382 and 482 partially overlap at the gastroesophageal junction 150b, in other embodiments, the first portions 382 and 482 may not overlap, and/or the second portions 384 and 484 may overlap at the greater curvature 136. In either case, the first and second constricting members 380 and 480 exert a force on the stomach 130 and/or gastroesophageal junction 150b to augment and/or imitate the tension applied by competent clasp fibers 162a to correct for the misalignment and altered state of the fibers 162b.

FIG. 11 is a schematic representation of a member 580 implanted relative to the stomach 130 of a patient 100b with GERD in accordance with another embodiment of the invention. The illustrated member 580 is positioned around the stomach 130 with a first portion 582 positioned at the angle of His 151 and a second portion 584 positioned at the greater curvature 136. The member 580 may be a constricting member that exerts a force on the stomach 130. Alternatively, the member 580 may be attached to the stomach 130 with sutures or other suitable means.

The illustrated member 580 further includes a plurality of electrodes 588, a power source 590 operably coupled to the electrodes 588, and a controller 592 operably coupled to the power source 590 for selectively energizing the electrodes 588. The constricting member 580 is generally aligned with the sling fibers 160b so that the electrodes 588 are positioned to stimulate the sling fibers 160b. Stimulation of the sling fibers 160b is expected to at least partially restores the gastroesophageal pressure gradient and enhances the mucosal seal of the lower esophageal sphincter 152b. In other embodiments, a second constricting member with a second plurality of electrodes can also be wrapped around the stomach 130 and aligned such that the second plurality of electrodes are positioned to electrically stimulate the sling and/or clasp fibers 160b and/or 162b.

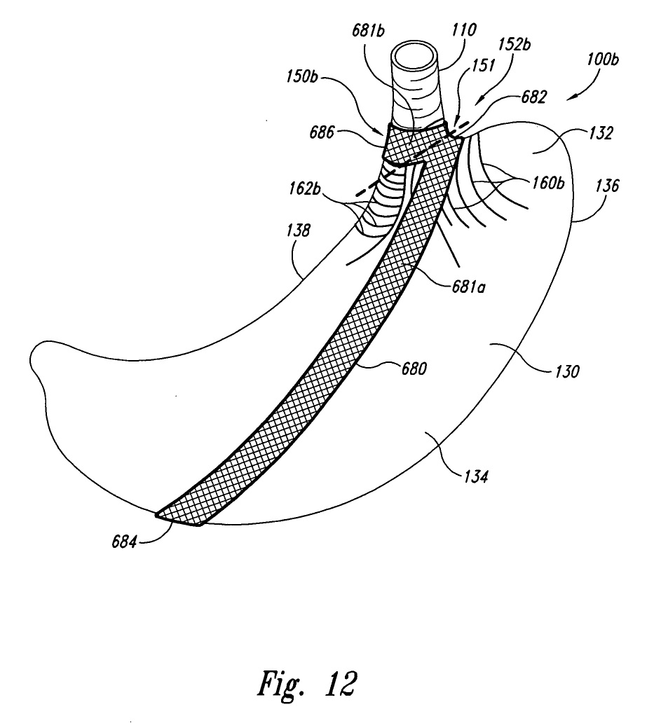

FIG. 12 is a schematic representation of a constricting member 680 implanted relative to the stomach 130 of a patient 100b with GERD in accordance with another embodiment of the invention. The illustrated constricting member 680 has a first segment 681a positioned around the stomach 130 and generally aligned with the sling fibers 160b and a second segment 681b positioned around the stomach 130 and generally aligned with the clasp fibers 162b. The first segment 681a includes a first portion 682 positioned at the angle of His 151 and a second portion 684 positioned at the greater curvature 136. The second segment 686 projects from the first portion 682 at the angle of His 151 and includes a portion 686 positioned at the gastroesophageal junction 150b. The first and second segments 681a-b of the constricting member 680 work together to exert a force on the stomach 130 and/or gastroesophageal junction 150b that augments and/or imitates the tension of competent sling and clasp fibers 160a and 162a to correct for the misalignment and altered state of the fibers 160b and 162b.

D. Embodiments of Methods for Implanting Constricting Members

The constricting members in the above-described embodiments can be implanted in a single surgical procedure. Most of the time, the constricting members can be placed by laparoscopic methods, which minimize the invasiveness of the surgery and reduce the duration of hospitalization. In some situations, however, a traditional open surgical method may be required. After accessing the abdominal cavity, the surgeon wraps the constricting member around the stomach and properly positions the member on the stomach. The end sections of the constricting member are then attached together with the proper degree of tension so that the member will exert the desired force on the stomach. The constricting member can be sutured or otherwise attached to the stomach so that the constricting member remains properly positioned on the stomach. Alternatively, the constricting member may not be sutured to the stomach, but rather the tension of the constricting member may be sufficient to hold the member in place.

From the foregoing, it will be appreciated that specific embodiments of the invention have been described herein for purposes of illustration, but that various modifications may be made without deviating from the spirit and scope of the invention. For example, many of the features of one embodiment can be combined with other embodiments in addition to or in lieu of the features of the other embodiments. Accordingly, the invention is not limited except as by the appended claims.

I/We claim:

1. A method of treating an esophageal disorder in a patient, the method comprising:
inserting a constricting member into the patient; and
positioning the constricting member along at least a portion of the stomach of the patient such that at least a section of the constricting member exerts a force on the stomach and/or gastroesophageal junction that improves the function of the lower esophageal sphincter.

2. The method of claim 1 wherein positioning the constricting member comprises placing a first portion of the constricting member at the angle of His and a second portion of the constricting member at the greater curvature of the stomach.

3. The method of claim 1 wherein positioning the constricting member comprises placing a first portion of the constricting member at the junction between the esophagus and the lesser curvature of the stomach and a second portion of the constricting member at the greater curvature of the stomach.

4. The method of claim 1 wherein positioning the constricting member comprises generally aligning the constricting member with at least some of the sling fibers of the lower esophageal sphincter.

5. The method of claim 1 wherein positioning the constricting member comprises generally aligning the constricting member with at least some of the clasp fibers of the lower esophageal sphincter.

6. The method of claim 1 wherein positioning the constricting member comprises placing the constricting member to increase the resting pressure of the lower esophageal sphincter.

7. The method of claim 1 wherein inserting the constricting member comprises implanting an inflatable bladder in the patient.

8. The method of claim 1 wherein inserting the constricting member comprises implanting an elastic band in the patient.

9. The method of claim 1 wherein inserting the constricting member comprises implanting an inelastic band in the patient.

10. The method of claim 1 wherein inserting the constricting member comprises implanting a band having a plurality of electrodes.

11. The method of claim 1, further comprising selectively adjusting the force exerted by the constricting member on the stomach and/or gastroesophageal junction.

12. The method of claim 1 wherein the constricting member comprises a first constricting member, wherein positioning the first constricting member comprises placing a first portion of the first constricting member at the angle of His and a second portion of the first constricting member at the greater curvature of the stomach, and wherein the method further comprises:
inserting a second constricting member into the patient; and
positioning the second constricting member around the stomach with a first portion of the second constricting member positioned at the angle of His and a second portion of the second constricting member positioned at the greater curvature, with the second constricting member at least partially spaced apart from the first constricting member.

13. The method of claim 1 wherein the constricting member comprises a first constricting member, wherein positioning the first constricting member comprises placing a first portion of the first constricting member at the angle of His and a second portion of the first constricting member at the greater curvature of the stomach, and wherein the method further comprises:
inserting a second constricting member into the patient; and
positioning the second constricting member around the stomach with a first portion of the second constricting member positioned at the junction between the esophagus and the lesser curvature of the stomach and a second portion of the second constricting member positioned at the greater curvature of the stomach.

14. The method of claim 1 wherein the constricting member comprises a first constricting member, wherein positioning the first constricting member comprises placing a first portion of the first constricting member at the junction between the esophagus and the lesser curvature of the stomach and a second portion of the first constricting member at the greater curvature of the stomach, and wherein the method further comprises:
inserting a second constricting member into the patient; and
positioning the second constricting member around the stomach with a first portion of the second constricting member positioned at the junction between the esophagus and the lesser curvature of the stomach and a second portion of the second constricting member positioned at the greater curvature of the stomach, with the second constricting member at least partially spaced apart from the first constricting member.

15. A method of treating an esophageal disorder in a patient, the method comprising implanting a band in the patient with the band positioned about a section of the stomach of the patient such that a portion of the band is located at the angle of His and exerts a force to increase the lower esophageal pressure.

16. The method of claim 15 wherein the portion of the band comprises a first portion, and wherein implanting the band further comprises positioning a second portion of the band at the greater curvature of the stomach.

17. The method of claim 15 wherein implanting the band comprises placing the band about the section of the stomach such that the band is generally aligned with the sling fibers of the lower esophageal sphincter.

18. The method of claim 15 wherein implanting the band comprises positioning the band to exert a force on the stomach and/or gastroesophageal junction and lengthen the high pressure zone of the lower esophageal sphincter.

19. The method of claim 15 wherein the band comprises a first band, wherein the portion of the first band comprises a first portion, wherein implanting the first band comprises placing a second portion of the first band at the greater curvature of the stomach, and wherein the method further comprises:
inserting a second band into the patient; and
positioning the second band around the stomach with a first portion of the second band positioned at the junction between the esophagus and the lesser curvature of the stomach and a second portion of the second band positioned at the greater curvature of the stomach.

20. The method of claim 15 wherein the band comprises a first band, wherein the portion of the first band comprises a first portion, wherein implanting the first band comprises placing a second portion of the first band at the greater curvature of the stomach, and wherein the method further comprises:
inserting a second band into the patient; and
positioning the second band around the stomach with a first portion of the second band positioned at the angle of His and a second portion of the second band positioned at the greater curvature of the stomach, with the second band at least partially spaced apart from the first band.

21. A method of treating an esophageal disorder in a patient, the method comprising:
implanting a reshaping member into the patient; and
positioning the reshaping member around a section of the stomach of the patient such that a first portion of the reshaping member is positioned between the esophagus and the fundus of the stomach and a second portion of the reshaping member is positioned at the greater curvature of the body of the stomach.

22. The method of claim 21 wherein positioning the reshaping member comprises placing the reshaping member so that the reshaping member exerts a force on the stomach and/or gastroesophageal junction to increase the resting pressure of the lower esophageal sphincter.

23. The method of claim 21 wherein positioning the reshaping member comprises placing the reshaping member so that the reshaping member exerts a force on the stomach and/or gastroesophageal junction to improve the competence of the lower esophageal sphincter.

24. The method of claim 21 wherein positioning the reshaping member comprises placing the reshaping member around the section of the stomach such that the reshaping member is generally aligned with the sling fibers of the lower esophageal sphincter.

25. The method of claim 21 wherein positioning the reshaping member comprises placing the first portion of the reshaping member at the angle of His to at least partially augment the tension applied by the sling fibers and improve the competence of the lower esophageal sphincter.

26. A method of treating an esophageal disorder in a patient, the method comprising implanting a band in the patient with the band positioned around a section of the stomach of the patient such that a first portion of the band is positioned at the junction between the esophagus and the lesser curvature of the stomach and a second portion of the band is positioned at the greater curvature of the stomach.

27. The method of claim 26 wherein implanting the band comprises positioning the band around the section of the stomach such that the band is generally aligned with at least some of the clasp fibers of the lower esophageal sphincter.

28. The method of claim 26 wherein implanting the band comprises positioning the band to exert a force on the stomach and/or gastroesophageal junction and improve the competence the lower esophageal sphincter.

29. The method of claim 26 wherein the band comprises a first band, and wherein the method further comprises:
inserting a second band into the patient; and
positioning the second band around the stomach with a first portion of the second band positioned at the junction between the esophagus and the lesser curvature of the stomach and a second portion of the second band positioned at the greater curvature of the stomach, with the second band at least partially spaced apart from the first band.

30. The method of claim 26 wherein the band comprises a first band, and wherein the method further comprises:
inserting a second band into the patient; and
positioning the second band around the stomach with a first portion of the second band positioned at the angle of His and a second portion of the second band positioned at the greater curvature of the stomach.

31. A method of treating an esophageal disorder in a patient, the method comprising:
inserting a constricting member into the patient; and
positioning the constricting member around a section of the stomach of the patient with at least a portion of the constricting member positioned at the junction between the esophagus and the lesser curvature of the stomach to exert a force that lengthens the high pressure zone of the lower esophageal sphincter.

32. The method of claim 31 wherein the portion of the constricting member comprises a first portion, and wherein positioning the constricting member comprises placing a second portion of the constricting member at the greater curvature of the stomach.

33. The method of claim 31 wherein positioning the constricting member comprises placing the constricting member around the section of the stomach such that the constricting member is generally aligned with the clasp fibers of the lower esophageal sphincter.

34. The method of claim 31 wherein positioning the constricting member comprises placing the constricting member to exert a force on the stomach and/or gastroesophageal junction and increase the lower esophageal sphincter pressure.

35. A method of treating an esophageal disorder in a patient, the method comprising:
accessing the abdominal cavity in the patient; and
a step for augmenting the contracting force of the lower esophageal sphincter muscles in the patient to increase the resting pressure of the lower esophageal sphincter.

36. The method of claim 35 wherein the step for increasing the contracting force comprises positioning a constricting member around at least a section of the stomach.

37. The method of claim 35 wherein the step for increasing the contracting force comprises placing a constricting member around at least a section of the stomach with a portion of the constricting member positioned at the angle of His.

38. The method of claim 35 wherein the step for increasing the contracting force comprises placing a constricting member around at least a section of the stomach with a first portion of the constricting member positioned at the angle of His and a second portion of the constricting member positioned at the greater curvature.

39. The method of claim 35 wherein the step for increasing the contracting force comprises placing a constricting member around at least a section of the stomach with a first portion of the constricting member positioned at the junction between the esophagus and the lesser curvature of the stomach and a second portion of the constricting member positioned at the greater curvature of the stomach.

40. The method of claim 35 wherein the step for increasing the contracting force comprises placing a constricting member around at least a section of the stomach such that the constricting member is generally aligned with at least some of the sling fibers of the lower esophageal sphincter.

41. The method of claim 35 wherein the step for increasing the contracting force comprises placing a constricting member around at least a section of the stomach such that the constricting member is generally aligned with at least some of the clasp fibers of the lower esophageal sphincter.

42. The method of claim 35 wherein the step for increasing the contracting force comprises:
placing a first constricting member around the stomach with a first portion of the first constricting member positioned at the angle of His and a second portion of the first constricting member positioned at the greater curvature; and
placing a second constricting member around the stomach with a first portion of the second constricting member positioned at the angle of His and a second portion of the second constricting member positioned at the greater curvature, with the second constricting member at least partially spaced apart from the first constricting member.

43. The method of claim 35 wherein the step for increasing the contracting force comprises:
placing a first constricting member around the stomach with a first portion of the first constricting member positioned at the angle of His and a second portion of the first constricting member positioned at the greater curvature; and
placing a second constricting member around the stomach with a first portion of the second constricting member positioned at the junction between the esophagus and the lesser curvature of the stomach and a second portion of the second constricting member positioned at the greater curvature.

44. The method of claim 35 wherein the step for increasing the contracting force comprises:
placing a first constricting member around the stomach with a first portion of the constricting member positioned at the junction between the esophagus and the lesser curvature of the stomach and a second portion of the first constricting member positioned at the greater curvature; and
placing a second constricting member around the stomach with a first portion of the second constricting member positioned at the junction between the esophagus and the lesser curvature of the stomach and a second portion of the second constricting member positioned at the greater curvature, with the second constricting member at least partially spaced apart from the first constricting member.

45. A band for treating an esophageal disorder in a patient, the band comprising a first portion configured to conform to the angle of His in the patient and a second portion configured to be positioned along a section of the stomach and overlay at least some of the sling fibers to improve the function of the lower esophageal sphincter.

46. The band of claim 45, further comprising an elastic member having the first and second portions.

47. The band of claim 45, further comprising an inelastic member having the first and second portions.

48. The band of claim 45, further comprising a plurality of electrodes for selectively stimulating at least some of the sling fibers.

49. The band of claim 45, further comprising an inflatable bladder having the first and second portions.

50. A band for treating an esophageal disorder in a patient, the band comprising a first portion configured to conform to the junction between the esophagus and the lesser curvature of the stomach and a second portion configured to conform to a section of the greater curvature of the stomach such that the band overlays at least some of the clasp fibers and exerts a force on the stomach to increase the lower esophageal pressure.

51. An apparatus for treating an esophageal disorder in a patient, the apparatus comprising:
a band having a first section configured to conform to the angle of His in the patient and a second section configured to be positioned at the greater curvature of the body of the stomach such that the band is at least generally aligned with at least some of the sling fibers in the patient; and
a plurality of electrodes attached to the band for selectively stimulating at least some of the sling fibers.

52. The apparatus of claim 51, further comprising a power source operably coupled to the electrodes and a controller operably coupled to the power source for selectively energizing the electrodes.

53. A band for treating an esophageal disorder in a patient, the band comprising:
a first segment with a first portion configured to conform to the angle of His and a second portion configured to conform to a first section of the greater curvature of the stomach; and
a second segment with a portion configured to conform to the junction between the esophagus and the lesser curvature of the stomach, wherein the second segment projects from the first segment at the first portion of the first segment.
